(19) United States
(12) Patent Application Publication
(10) Pub. No.: US 2024/0148950 A1
(43) Pub. Date: May 9, 2024

(54) DIALYSIS CATHETER CLEARANCE DEVICE AND ASSOCIATED METHOD

(71) Applicant: Stanley Batiste, Granite Bay, CA (US)

(72) Inventor: Stanley Batiste, Granite Bay, CA (US)

(21) Appl. No.: 18/506,040

(22) Filed: Nov. 9, 2023

Related U.S. Application Data
(63) Continuation-in-part of application No. 17/211,741, filed on Mar. 24, 2021.
(60) Provisional application No. 63/424,438, filed on Nov. 10, 2022.

(51) Int. Cl. A61M 1/36

(52) U.S. Cl. CPC A61M 1/3661

(57) ABSTRACT

A dialysis catheter clearance device is for use with a dialysis catheter. The device includes an infusion catheter having a first end, a second end, and a lumen between the first end and the second end. The device also includes a flexible rod, within the infusion catheter. The rod has a first end and a second end. The first end of the rod is connected to a motor, which imparts movement into the rod, and the second end of the rod is located at or about the second end of the infusion catheter. Actuation of the motor imparts movement to the second end of the rod to induce turbulence in clot dissolving medication, thereby increasing the effectiveness of the clot dissolving medication in dissolving a clot located at the second end of the infusion catheter.

### BACKGROUND OF THE INVENTION

### 1. Field of Invention

The present invention relates to clearance of occluded catheters.

### 2. Description of Related Art

Patients with end stage renal disease (“ESRD”) have lost their normal kidney function, and as a result require dialysis to substitute the function of the kidney cleansing the blood. ESRD affects almost 750,000 people per year in the United States. Hemodialysis requires that large volume blood access and exchange be consistently available to sustain the life of the patient. Medicare coverage is extended to a person of any age who requires either dialysis or transplantation to maintain life. The people who live with ESRD are 1% of the U.S. Medicare population but account for roughly 7% of the Medicare budget. Mortality rates vary depending on the ESRD treatment. After one year of treatment, those on dialysis have a 20-25% mortality rate, with a 5-year survival rate of 35%. Persons who receive transplants have a 3% mortality rate after 5 years. There are two types of dialysis, hemodialysis, and peritoneal dialysis. For purposes of this overview, we will primarily be focused on hemodialysis.

Hemodialysis care costs the Medicare system an average of $90,000 per patient annually in the United States, for a total of $28 billion. Typically, a dialysis patient will require 3-4 hours of dialysis three days a week. The challenge with providing hemodialysis is maintaining access to large volumes of blood when a body constantly fights attempts to keep access available by healing closed such access. Currently there are three ways to provide hemodialysis; dialysis catheters, arterial venous fistulas (AVF's) and arterial venous grafts (AVGs). Although used worldwide, catheters are known not to be efficient for long term dialysis. Unfortunately, catheters have very short patency rates and high rates of infection greater than 60% of all dialysis patients use catheters.

Long term catheter patency rates remain low at less than 35% after 1 year and an average patency rate of 80 days. It is the development of a fibrin sheath that determines the long term patency of a catheter. This sheath, initially composed of fibrinogen, albumin, lipoproteins, and coagulation factors, begins to form within 24 hours of insertion. The fibrin sheath attracts platelets and coagulation factors and promotes leukocyte adherence. Over weeks and months, collagen is deposited as smooth muscle cells from the venous vessel wall migrate toward the tip. The rate of these processes varies among patients because of inherited or acquired characteristics. Ultimately, if clotting in excess of the endogenous fibrinolytic system's capacity develops, catheter thrombosis occurs.

There are several ways to restore patency to an existing catheter if it is decided that a new catheter placement at a different site may be delayed. Commonly, a catheter may be exchanged for a new catheter using guidewires as placeholders when the initial catheter is removed. The guidewires are generally advanced using fluoroscopic guidance, the catheter is then liberated from the body tissues and a new catheter is then advanced over the guidewire to the same location as the prior, occluded catheter. This method, although effective, requires patient sedation, access to a surgical or fluoroscopic suite and numerous hospital personnel, including at least one nurse and a physician. The major setback is that the catheter follows in the same tract as the prior catheter, and it may be directed into the same fibrin sleeve that has formed.

### SUMMARY

Dialysis catheter occlusion is a common problem affecting nearly every hemodialysis patenting who has one. Overall catheter patency rates are low, and catheter use in our system remains high creating increased healthcare costs and significant frustrations for those dialysis patients. A catheter occlusion will generally be discovered at the dialysis center and many times patients will need to go to the hospital for treatment prior to receiving dialysis. Once at the hospital thrombolytic medication can be injected at the entry port or patient can have the surgical or Interventional radiology teams exchange the catheter while sedated. While these methods have shown some success and are currently employed to restore patency, the described invention and method of use creates a much improved means of using the thrombolytic that speeds up lyses times and improved fibrin and clot removal.

The fibrin which can form all along the catheter causes occlusion once the fibrin sheath covers the distal tip. Generally, the inflow port of the catheter will be useable as an injection will displace the fibrin and allow fluid passage out of the catheter. The entry/blood aspiration/draw port however remains non-functional as the fibrin acts as a ball-valve mechanism not allowing blood to flow to the proximal catheter. The inner lumen volume of the dialysis catheter may be upwards of 2 ccs in each port. When thrombolytics are injected they diffuse through the 2 ccs and some of it reaches the tip and goes on to lyse the fibrin. Much of the thrombolytics however remains unused within the length of the catheter not coming into contact with the fibrin at the tip. The described invention is an innovative means of applying the thrombolytics directly at the catheter tip and can be utilized at the patient's bedside without the need for surgical suite or a large medical team.

A non-invasive means of restoring patency to a catheter is that of employing lytic therapy which has proven effective. This is performed by using a syringe to inject a thrombolytic medication such as TPA (Tissue Plasminogen Activator) directly into the proximal port of the catheter and allowed to “soak” in the catheter lumen to dissolve the fibrin sheath at the tip. This may be performed without use of imaging requiring only a nurse to perform. After 1-3 hrs., the catheter is checked for patency by aspiration using a syringe. The invention described relates to thrombolysis of catheter using more directed thrombolytic therapy.

The described innovation utilizes an intraluminal catheter placed with in the lumen of the dialysis catheter to apply directed thrombolysis at the tip where the largest thrombus burden exists. The catheter is created in specific sizes, or in one embodiment—a variable size in order to provide direct infusion. The design allows the user to match the need infusion length with the dialysis catheter size and precisely direct drug infusion at the exact point of need. The application can be performed in a non-surgical setting such as the ED or in the dialysis clinic with the need for only a chest x-ray for placement confirmation.

Although prior art describes the use of catheters for thrombolysis the presented invention creates a means to exploit the standard design of dialysis catheters in order to allow the user to apply the drug in precise location at the patient's bedside or in an outpatient setting such as a dialysis center. To further the utility of the invention means of length and quantity of drug administration are combined as the art combines and infusion module with the measured infusion catheter. The device uses either preloaded medication, or in a second embodiment, the medication is added to the device prior to its use. The invention is used as a disposable, self-contained system which can be matched to the appropriate dialysis catheter taken out of its packaging and either loaded with medication or preloaded then advanced into the patient's catheter, adjusted for medication duration and then turned on. Once the medication has been given, the catheter and system are removed, and the patient can then be dialyzed.

Other systems, methods, features, and advantages of the invention will be or will become apparent to one with skill in the art upon examination of the following figures and detailed description. It is intended that all such additional systems, methods, features, and advantages be included within this description, be within the scope of the invention, and be protected by the accompanying claims. Further, any of the elements and features disclosed herein may be combined in any manner with any of the other elements and features disclosed herein.

### DESCRIPTION OF THE DRAWINGS

The components in the figures are not necessarily to scale, emphasis instead being placed upon illustrating the principles of the invention. In the figures, like reference numerals designate corresponding parts throughout the different views

FIG. 1 illustrates a standard tunneled type dialysis catheter.

FIG. 2 illustrates a dialysis catheter with the distal tip positioned near the heart in the chest.

FIG. 3 illustrates an occluded dialysis catheter with development of fibrin and clot at the distal tip.

FIG. 4 illustrates an injection of medication into the dialysis catheter port with medication diffusion to the tip.

FIG. 5 illustrates the infusion system with the infusion pump and control 100 and the preset infusion catheter 200 and the associated components.

FIG. 6 illustrates an anti-leak connector 300 with internal Luer lock connector 310 and anti-leak valve 320 sealing around the infusion catheter 200.

FIG. 7 illustrates an infusion system deployed with the tip in the dialysis catheter and medication infused directly at the dialysis catheter tip.

FIG. 8 illustrates the infusion system deployed within a longer dialysis catheter confirmed by the matched infusion catheter marker 211.

FIG. 9 illustrates the lysis process, from left to right wherein the medication 50 dissolves the fibrin and clot 4 and restored catheter patency.

FIG. 10 illustrates an inside view of the infusion pump system 101 with internal components visualized.

FIG. 11 illustrates the components housed within the Infusion system 100, a medication reservoir 150 and output catheter 190 and the infusion controls 120 with electronically controlled motor and pump mechanism 170 and 175.

FIG. 12 illustrates a flow rate control 130 sensory set on low with minimal medication 50 output.

FIG. 13 illustrates a flow rate control 130 sensory set on medium with moderate medication 50 output.

FIG. 14 illustrates a flow rate control 130 sensory set on high with maximum medication 50 output.

FIG. 15 illustrates an additional embodiment of the infusion system with a predetermined, non-adjustable catheter length and anti-leak connecter.

FIG. 16 illustrates an additional embodiment of the infusion system with two outflow catheters.

FIG. 17 illustrates an additional embodiment of the infusion system with measurement markers along the proximal infusion catheter.

FIG. 18 illustrates an infusion system that is preloaded with a dose of the medication therefore not requiring the infusion port.

FIG. 19 illustrates an infusion system with minimally compliant drug reservoir and outflow resister unit.

FIG. 20 illustrates and infusion system with minimally compliant drug reservoir and an engaged outflow resister unit compressing outflow tubing.

FIG. 21 illustrates a detachable coupling unit separating the catheter from the infusion system.

FIG. 22 illustrates a detachable coupling unit and catheter detached from the infusion system Luer lock port as well as a universal catheter coupling attached to the infusion system Luer lock for various types of catheters.

FIG. 23 illustrates a balloon tip in communication with a catheter and a Luer lock engaged by a syringe.

FIG. 24 illustrates an inflated balloon tip when a syringe is depressed to inflate the balloon tip.

FIG. 25 illustrates a balloon tip deflated and then retracted into a catheter for proper placement prior to drug infusion.

FIG. 26 illustrates a distal balloon with distal infusion capability attached to a syringe.

FIG. 27 illustrates an inflated distal balloon, inflated through use of a syringe, extending from a distal end of a catheter.

FIG. 28 illustrates an infusion catheter tip, at the distal end of a dialysis catheter, show as deflated, and then inflated, to provide directional drug infusion.

FIG. 29 illustrates a barbed tip at the distal end of an infusion catheter which allows tactile resistance in catheter positioning.

FIG. 30A, FIG. 30B, FIG. 30C, and FIG. 30D illustrate the barbed tip of the infusion catheter positioned at various locations distal to the dialysis catheter.

FIG. 31 is simplified view of a dialysis catheter clearance device, in accordance with one non-limiting embodiment of the disclosed concept.

FIG. 32 is an enlarged view of a portion of the dialysis catheter clearance device of FIG. 31.

FIG. 33 is an enlarged view of another portion of the dialysis catheter clearance device of FIG. 31.

FIG. 34 is an enlarged view of a portion of another dialysis catheter clearance device, in accordance with another non-limiting embodiment of the disclosed concept.

FIG. 35 is an enlarged view of another portion of the dialysis catheter clearance device of FIG. 34.

FIG. 36 is a simplified view of another dialysis catheter clearance device, in accordance with another non-limiting embodiment of the disclosed concept.

FIG. 37 is a simplified view of another dialysis catheter clearance device, in accordance with another non-limiting embodiment of the disclosed concept.

### DETAILED DESCRIPTION

Hemodialysis patients require routine large volume blood exchange to survive, and our bodies fight off efforts to allow this access. For many dialysis patients a permanent, indwelling catheter is the means of providing such access. Dialysis catheters have advantages over other methods of access however also have a limited time in which they will stay open and function mainly because of fibrin and clot forming on the tip. This invention and the method of use describe a means to direct a drug, a thrombolytic, directly at the point needed for a specific duration for dialysis catheters which are made in specific standard sizes.

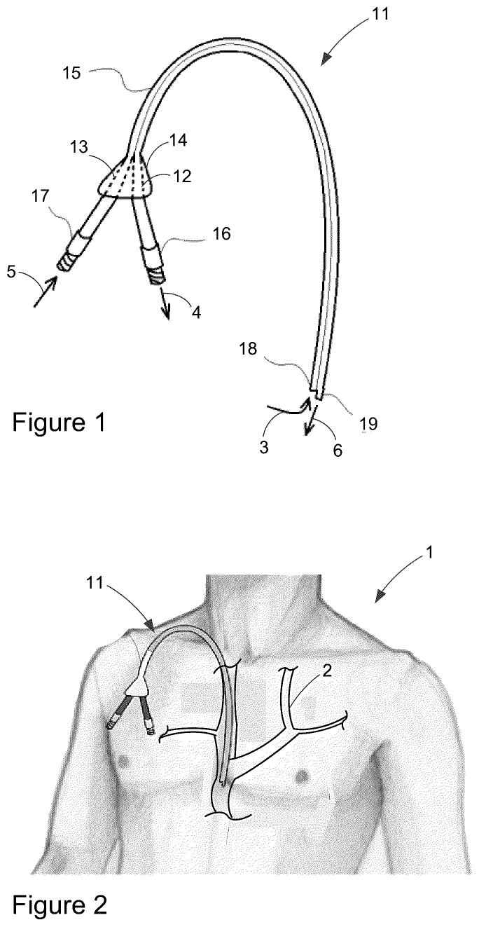

FIG. 1 illustrates a standard dialysis catheter 11 consisting of an aspiration tube 12, an injection tube 13 held together by a cuff 14 and a catheter 15. The aspiration tube 12 is attached to an aspiration port 16 on one end, and a distal tip on the other end 18. The injection tube 13 is attached to an injection port 17 on one end, and a distal tip on the other end 19. The arrows 3, 4, 5, 6 represent the direction of the blood flow. Specifically, the patient's blood exits the patient's body by entering 3 the distal tip of the aspiration tube 12, flowing through the aspiration tube 12, and exiting 4 the aspiration port 16 to become filtered. Once filtered, the patient's blood is returned to the patient's body by entering 5 through the injection port 17, flowing through the injection tube 13, and exiting 6 the distal tip of the injection tube 19.

FIG. 2 illustrates the standard dialysis catheter 11 implanted in a patient's 1 chest near the heart, with the distal tips of the aspiration tube 18 and the injection tube 19 inserted into the patient's chest 1 superior vena cava blood vessel 2.

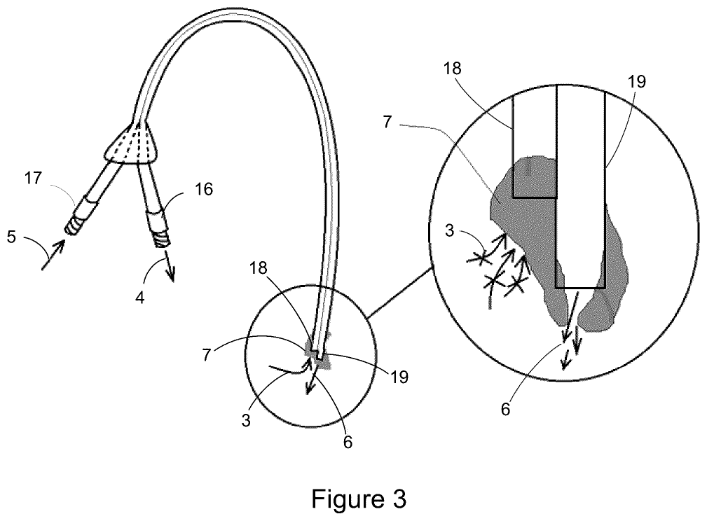

FIG. 3 illustrates how the fibrin and clot 7 developed on the distal tips of the aspiration tube 18 and the injection tube 19 restricting the flow of the patient's blood through the standard dialysis catheter 11. The patient's blood exiting 6 the distal tip of the injection tube 19 may be able to break the fibrin and clot 7 forming around the distal tip of the injection tube 19 due to pressure. However, the patient's blood entering 3 the distal tip of the aspiration tube 18 cannot pass through the fibrin and clot 7 forming around the distal tip of the aspiration tube 18 due to the ball-valve mechanism.

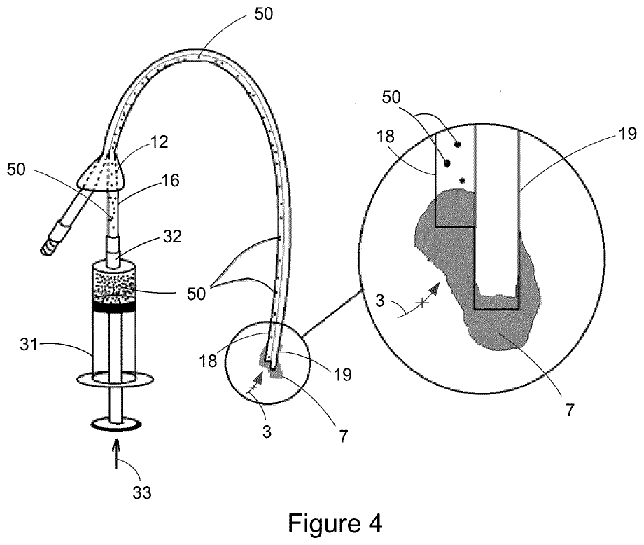

FIG. 4 illustrates the standard method of restoring patency in the standard dialysis catheter 11. A syringe 31 containing medication 50 attaches to the aspiration port 16 using a standard Luer lock attachment 32. The syringe 31 injects 33 the medication 50 in the aspiration port 16. Pressure from the injection 33 pushes the medication 50 into the aspiration tube 12. This method intends the medication 50 to reach the distal tip of the aspiration tube 18, where the medication 50 breaks down the fibrin and clot 7 to allow the patient's blood to enter 3 the distal tip of the aspiration tube 18. However, this method is ineffective because the injection 33 does not produce enough pressure, resulting in most of the injected medication 50 to remain in the aspiration tube 12 without reaching the distal tip of the aspiration tube 18. The small amount of medication 50 reaching the distal tip of the aspiration tube 18 is not sufficient to break down enough fibrin and clot 7 to allow the patient's blood to enter 3 the distal tip of the aspiration tube 18.

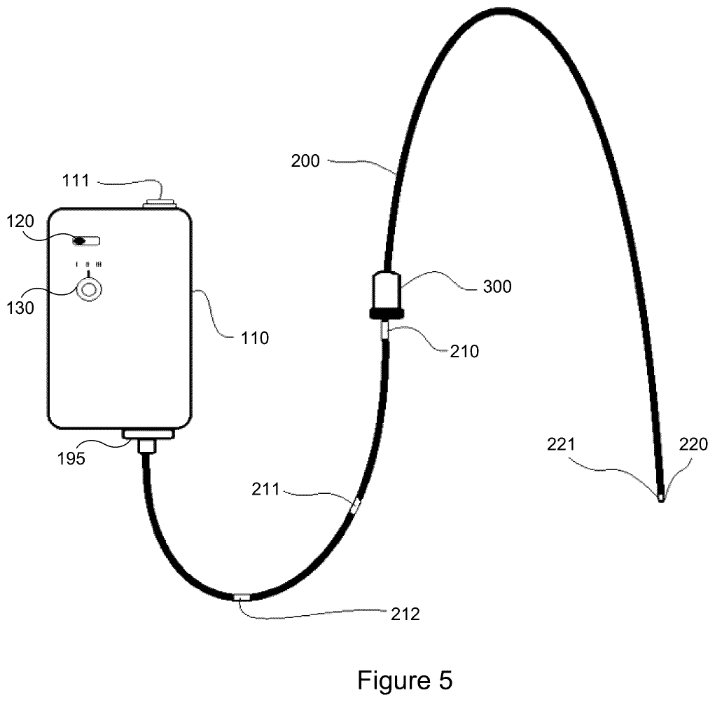

FIG. 5 illustrates an embodiment of the catheter clearance device 100, consisting of a catheter clearance box 110 connected to a medication injection port 111 on one end, and an infusion catheter connector 195 on the other end. The infusion catheter connector 195 connects the catheter clearance box 110 to an infusion catheter 200.

The infusion catheter 200 is connected to the catheter clearance box 110 on one end and has a distal tip 220 on the other end. A connector and valve 300 attaches to the infusion catheter 200. The infusion catheter 200 displays placement markers measuring 19 centimeters 210, 23 centimeters 211, and 27 centimeters 212 respectively from the distal tip 220 of the infusion catheter 200. The distal tip 220 of the infusion catheter 200 contains a radiopaque marker 221, which can be detected by x-ray.

A port switch 120 on the catheter clearance box 110 turns the catheter clearance device 100 on and off. A flow rate control selector 130 on the catheter clearance box 110 controls the speed at which medication travels from the catheter clearance box 110 to the distal tip 220 of the infusion catheter 200.

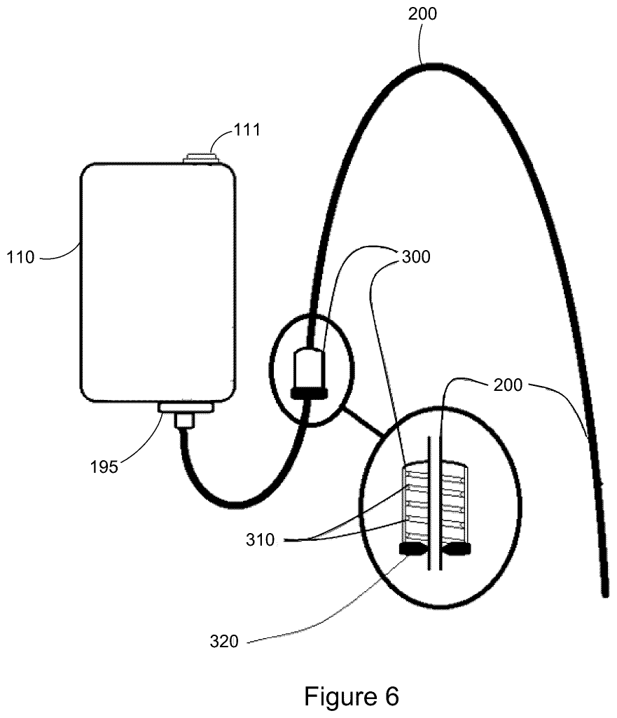

FIG. 6 illustrates the connector and valve 300, which is attached to the infusion catheter 200 and can be moved along the length of the infusion catheter 200 for proper placement of the infusion catheter 200 inside standard dialysis catheters. The connector and valve 300 consist of a female Luer lock connector 310 and an anti-leak valve 320. The female Luer lock connector 310 can attach to the aspiration port 16 of a standard dialysis catheter 11. The anti-leak valve 320 can seal the outer portion of the infusion catheter 200. When fully sealed, the anti-leak valve 320 prevents the flow of medication inside the infusion catheter 200 from escaping, thus directing the medication to the clot.

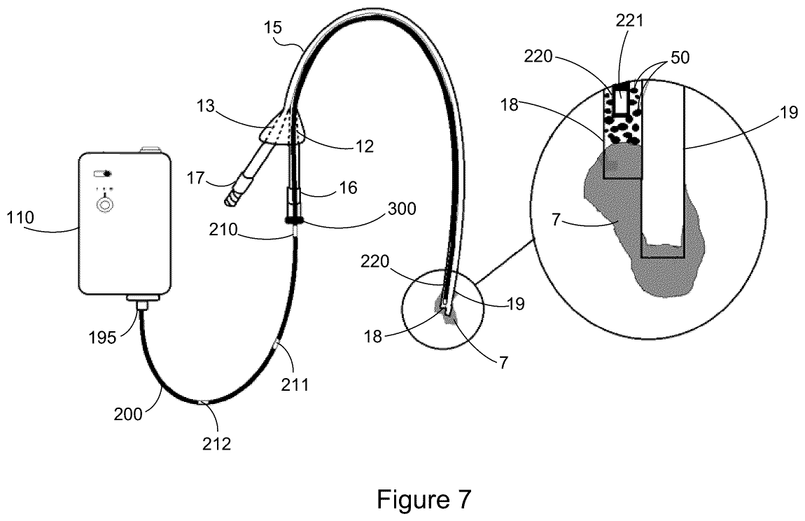

FIG. 7 illustrates the placement of the catheter clearance device 100 inside the aspiration tube 12 of a standard dialysis catheter 11. Specifically, the infusion catheter 200 is inserted into the catheter 15 of the standard dialysis catheter 11 through the aspiration port 16. The catheter clearance device 100 is correctly placed inside a standard dialysis catheter 11 when the distal tip 220 of the infusion catheter 200 reaches the distal tip of the aspiration tube 18. In this illustration, the first placement marker 210 confirms this correct placement when the placement marker 210 is positioned directly underneath the aspiration port 16 of the standard dialysis catheter 11. The correct placement can also be confirmed by an x-ray showing the radiopaque marker 221 is aligned with the distal tip of the aspiration tube 18 of the standard dialysis catheter 11. The connector and valve 300 attaches to the aspiration port 16 to secure the infusion catheter 200 inside the catheter 15 of the standard dialysis catheter 11 once correct placement is confirmed. The catheter clearance device 100 can also be placed inside the injection tube 13 of the standard dialysis catheter 11 using the same method described above.

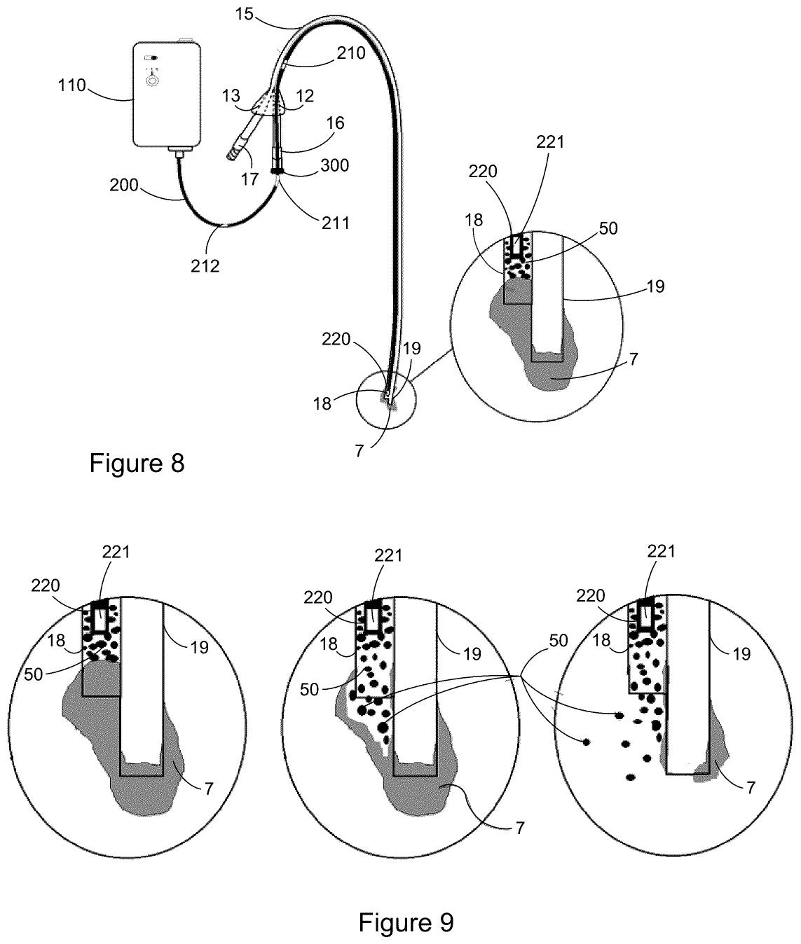

FIG. 8 illustrates the placement of the catheter clearance device 100 inside a standard dialysis catheter 11 with a longer catheter 15, such that the infusion catheter 200 is advanced until the second placement marker 211.

FIG. 9 the infusion of the medication 50 to the distal tip of the aspiration tube 18 of the standard dialysis catheter 11. The distal tip 220 of the infusion catheter 200 ensures delivery of all injected medication 50 directly to the distal tip of the aspiration tube 18. Thus, there will be sufficient medication 50 to dissolve the fibrin and clot 7, restoring patency to the standard dialysis catheter 11.

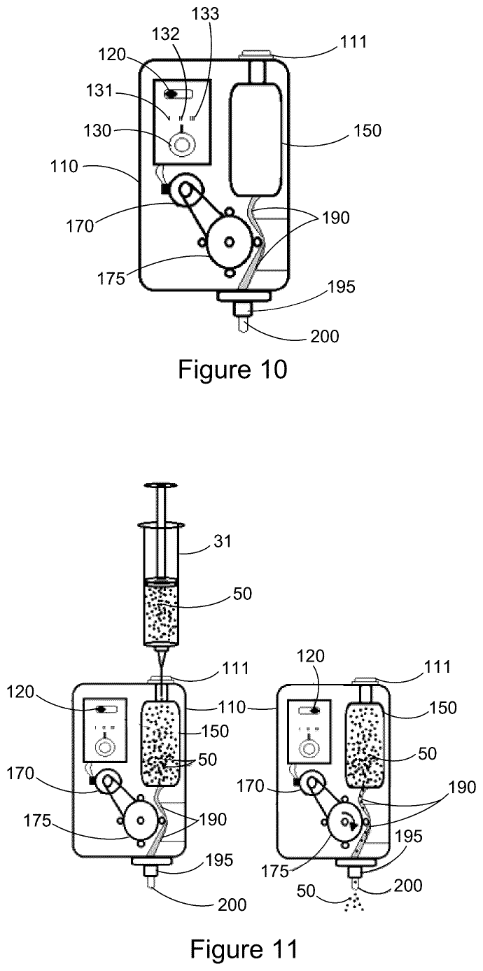

FIG. 10 illustrates the interior of the catheter clearance box 110. The medication injection port 111 at the top of the catheter clearance box 110 is connected to a medication reservoir 150. In one embodiment of the invention, the medication reservoir 150 is made of an evacuated compliant sac structure that expands as it accepts fluids. This embodiment eliminates air in the system. The other end of the medication reservoir 150 is connected to an outflow line 190. The other end of the outflow line 190 is connected to the infusion catheter connector 195. The infusion catheter 200 attaches to the other end of the infusion catheter connector 195. The port switch 120 is connected to the motor 170. The motor is connected to the belt and infusion gear 175.

The flow rate control selector 130 displays three flow rate options on the exterior of the catheter clearance box 110. The three respective flow rate options are minimum 131, medium 132, and maximum 133. On the interior of the catheter clearance box 110, the flow rate control selector 130 is connected to the motor 170.

FIG. 11 illustrates how medication 50 travels through the catheter clearance box 110. Specifically, a syringe 31 injects 33 medication 50 through the medication injection port 110. The medication 50 then flows into the medication reservoir 150.

When the port switch 120 is on, the motor 170 powers the belt and infusion gear 175 to rotate. The belt and infusion gear 175 pushes the medication 50 in the medication reservoir 150 through the outflow line 190 to the infusion catheter connector 195, where the medication 50 flows into the infusion catheter 200.

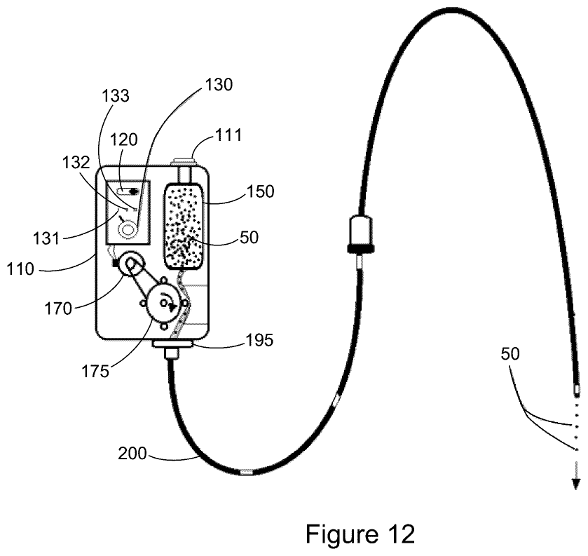

FIG. 12 illustrates the catheter clearance device 100 operating on minimum 131. Specifically, when the flow rate control selector 130 is set to minimum 131 and the port switch 120 is on, the motor 170 powers the belt and infusion gear 175 that rotates at a slow speed. As a result, the medication in the medication reservoir 150 is slowly pushed into the outflow line 190, then through the infusion catheter connector 195 into the infusion catheter 200, eventually reaching the distal tip 220 of the infusion catheter 200 and exiting the infusion catheter 200 at a slow rate.

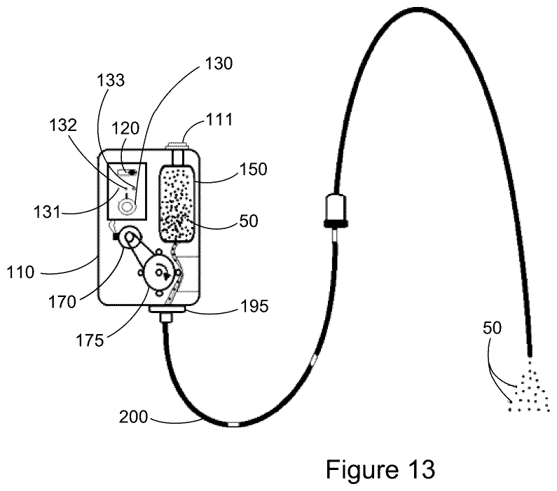

FIG. 13 illustrates the catheter clearance device 100 operating on medium 132. Specifically, when the flow rate control selector 130 is set to medium 132 and the port switch 120 is on, the motor 170 powers the belt and infusion gear 175 that rotates at a medium speed. As a result, the medication in the medication reservoir 150 is pushed into the outflow line 190 at a medium speed, then through the infusion catheter connector 195 into the infusion catheter 200, eventually reaching the distal tip 220 of the infusion catheter 200 and exiting the infusion catheter 200 at a medium rate. The medication may exit the infusion catheter through a nozzle, which may comprise any type opening at the distal end of the infusion catheter through which medication pass out of the infusion catheter 200.

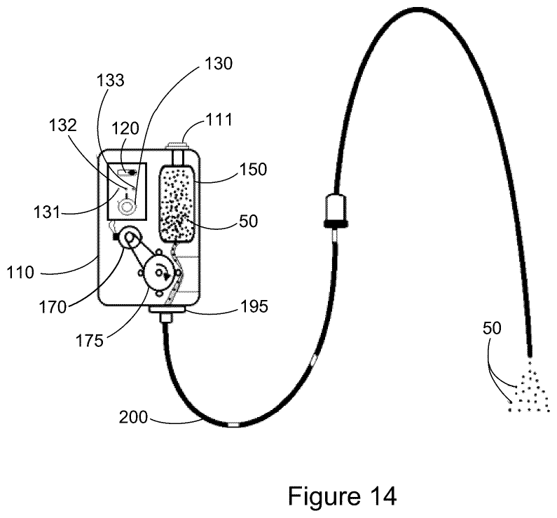

FIG. 14 illustrates the catheter clearance device 100 operating on maximum 133. Specifically, when the flow rate control selector 130 is set to maximum 133 and the port switch 120 is on, the motor 170 powers the belt and infusion gear 175 that rotates at a high speed. As a result, the medication in the medication reservoir 150 is pushed into the outflow line 190 at a high speed, then through the infusion catheter connector 195 into the infusion catheter 200, eventually reaching the distal tip 200 of the infusion catheter 200 and exiting the infusion catheter 200 at a fast rate.

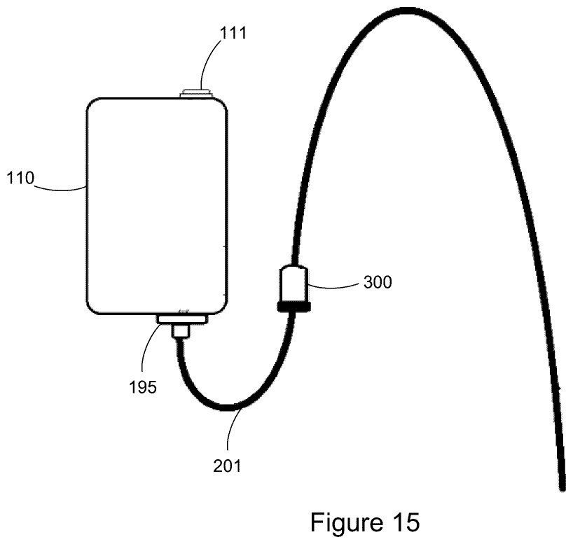

FIG. 15 illustrates another embodiment of the catheter clearance device 100 using a premeasured infusion catheter 200, which does not display placement markers 210, 211, 212. The placement of the connector and valve 300 is preset such that the connector and valve 300 cannot move along the infusion catheter 200. Such infusion catheters 200 vary in length and are based on the length of catheters on standard dialysis catheters 11. Using this embodiment, the user would choose the appropriate length of a premeasured infusion catheter 200 to insert into the infusion catheter connector 195.

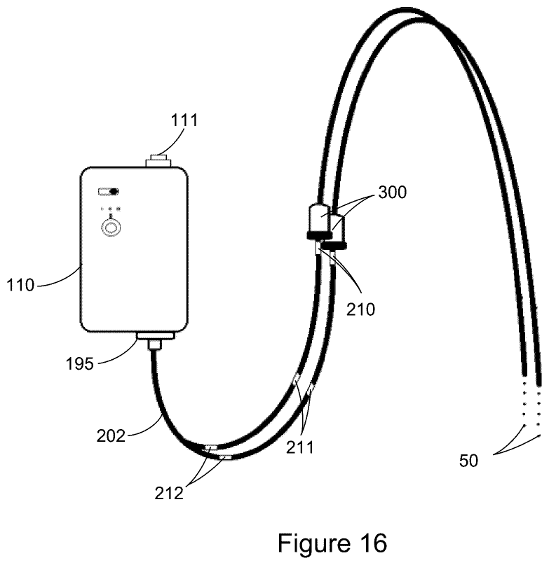

FIG. 16 illustrates another embodiment of the catheter clearance device 100 where a 2-prong infusion catheter 202 is attached to the infusion catheter connector 195. The 2-prong infusion catheter 202 splits into two tubes, each tube displaying three placement markers 210, 211, 212. A connector and valve 300 are attached to each tube. This embodiment allows the catheter clearance device 100 to be placed inside both the aspiration tube 12 and the injection of a standard dialysis catheter 11, such that a single catheter clearance device 100 can remove fibrin and clot 7 at the distal tips of the aspiration tube 18 and the injection tube 13 of the standard dialysis catheter 11 simultaneously.

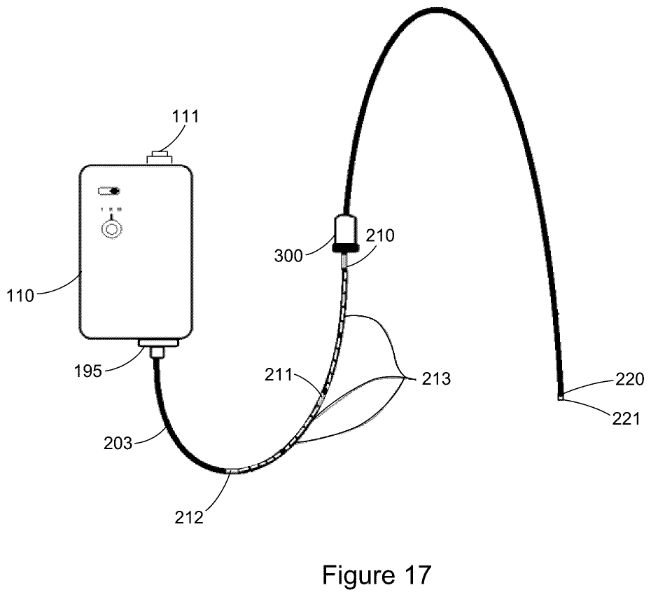

FIG. 17 illustrates another embodiment of the catheter clearance device 100 where an Infusion catheter with measuring marks 203 is attached to the infusion catheter connector 195. In this embodiment, measuring marks 213 are displayed between the placement markers 210, 211, 212 such that the catheter clearance device 100 can restore the patency of dialysis catheters with non-standard length catheters. The measuring marks 213 may also help adjust the placement of the infusion catheter 200 inside the catheter 15 of a standard dialysis catheter 11, should an x-ray of the radiopaque marker 221 indicate the radiopaque marker 221 is not fully aligned with the distal tip of the aspiration tube 18 or the injection tube 13 of a standard dialysis catheter 11.

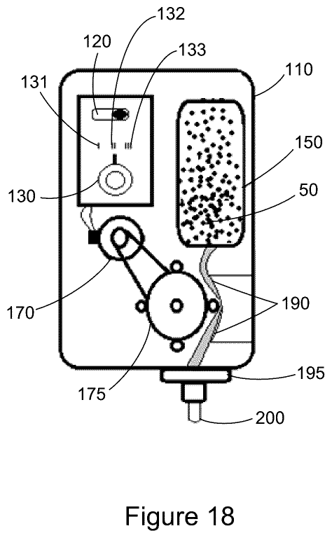

FIG. 18 illustrates another embodiment of the catheter clearance device 100 with no medication injection port 111 on the catheter clearance box 110. In this embodiment, the medication reservoir 150 is preloaded with medication 50. This embodiment allows the user to utilize the catheter clearance device 100 without the need to manually inject 33 medication 50 into the catheter clearance box 110.

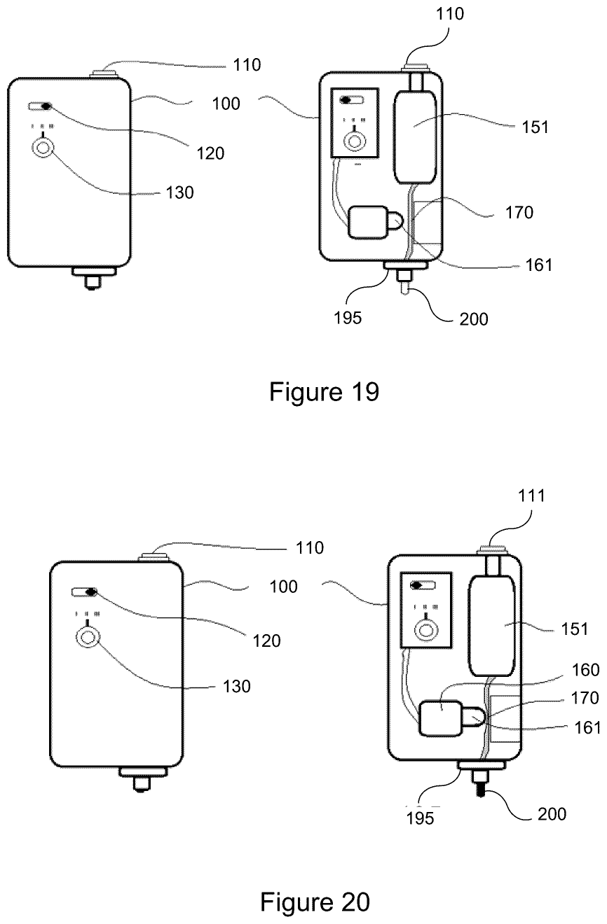

Also disclosed herein are additional embodiments and methods of use. FIG. 19 illustrates the infusion system wherein the minimally compliant drug reservoir 151 is configured to accept an injection through the injection hub 110 which expands the reservoir 151 which is then under pressure. Identical reference numbers identify similar elements. The drug then travels from the reservoir 151 through the outflow tubing 170 bypassing the outflow resistor plunger 161 and continues into the outflow catheter. In this embodiment, the pump or pusher system may or may not be used. Due to the elastic nature of the reservoir 151, this embodiment will operate without power. The reservoir 151 may have a flow limiting device at its outflow opening which connects the outflow tubing 170. The injection hub 110 may be self-sealing to maintain the reservoir 151 free from contamination and maintain pressure in the reservoir.

FIG. 20 illustrates and infusion system with minimally compliant drug reservoir and an engaged outflow resister unit compressing outflow tubing. As shown, the outflow resistor plunger 161 is engaged to contact the outflow tubing 170 thereby providing pressure on the outflow tubing creating resistance slowing and ultimately stopping drug flow through the infusion system. The plunger 161 may be controlled by the flow rate control selector/sensor 130 such that the more the plunger is pushed into the tubing, the less flow is possible and with sufficient plunger movement, flow is entire stopped. The plunger 161 may activate periodically to control flow over time, such as for example, ten minutes every hour, or any other time intervals. The outflow resistor plunger 161 may be controlled by the system of switches 120, 130 and the outflow resistor unit 160. The plunger 161 may be movement controlled by a solenoid, stepper motor, or any other control mechanism. In other embodiments, structures other than a plunger as shown may be used.

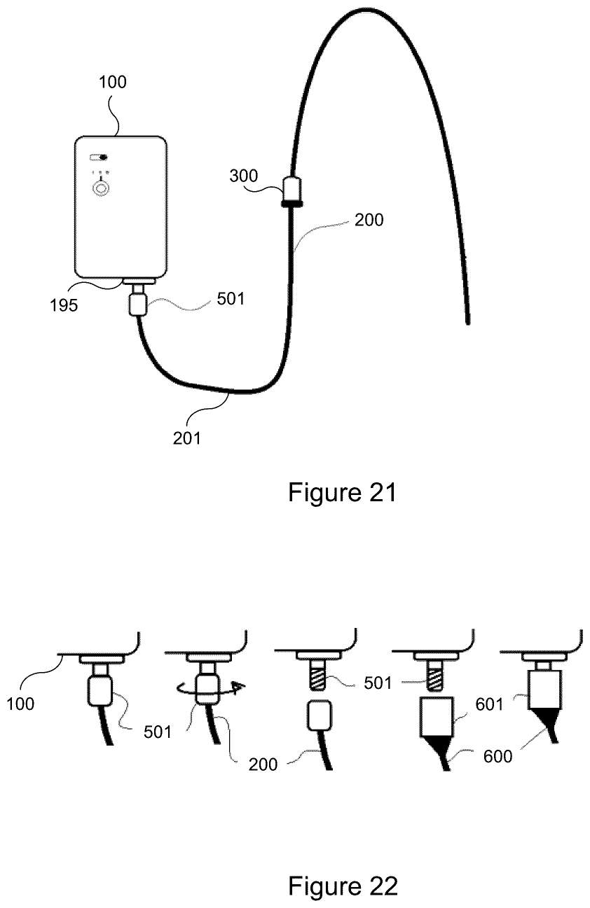

FIG. 21 illustrates a detachable coupling unit separating the catheter from the infusion system. FIG. 22 illustrates a detachable coupling unit and catheter detached from the infusion system Luer lock port as well as a universal catheter coupling attached to the infusion system Luer lock for various types of catheters. FIGS. 21 and 22 illustrate an embodiment having the detachable coupling unit (501) which allows use of any standard catheter with a universal coupling (601) Luer lock type of connector. This allows the user to attach the infusion system directly to an indwelling catheter and perfuse the medication from the proximal end to the distal end without the need for a separate inter-luminal infusion catheter. Although described as a Luer lock type connector, any type or configuration of connector may be used.

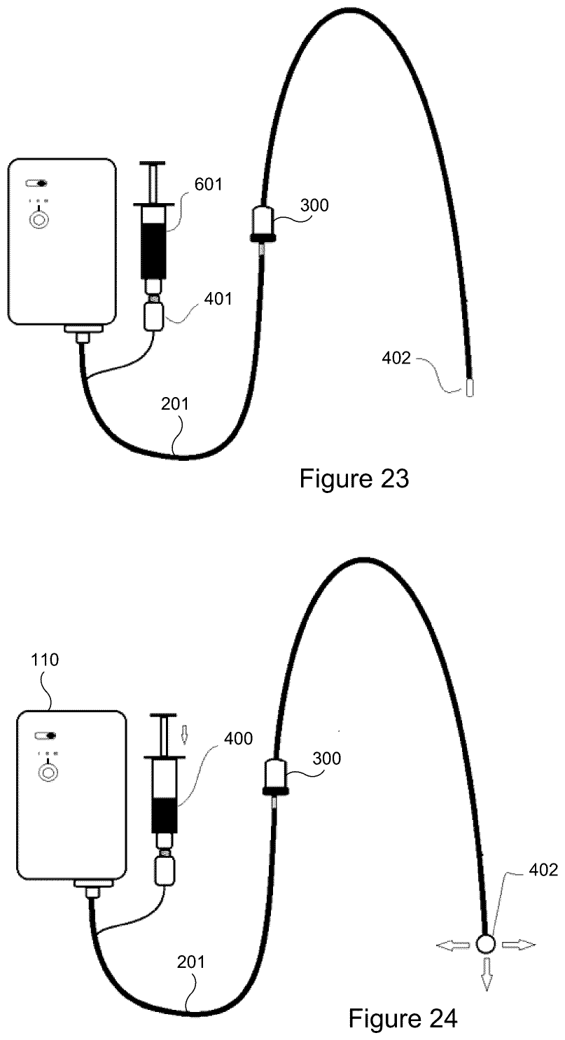

FIG. 23 illustrates a balloon tip in communication with a catheter and a Luer lock engaged by a syringe. As shown, a syringe 400 is filled with a liquid (or air) and in fluid communication with a balloon 402 which is advanced through the infusion catheter 200 to a distal end of the catheter to be just outside of the catheter. The distal tip of the balloon 402 may be advanced into the catheter and further beyond the distal tip by several centimeters. It may be difficult for a user, such as a doctor or nurse, to know the location of the balloon in relation to the end of the infusion catheter 200.

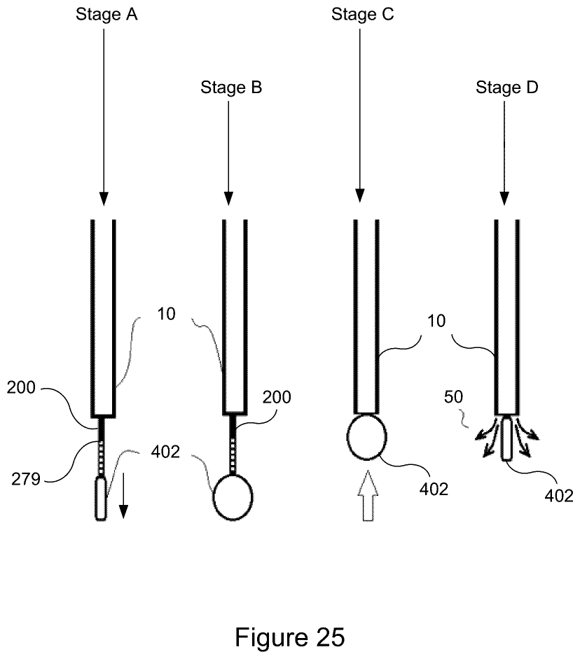

FIG. 24 illustrates an inflated balloon tip when a syringe is depressed to inflate the balloon tip. Depressing the syringe 400 causes the fluid in the syringe to flow into the balloon 402 to thereby inflate the balloon. The balloon 402 may be any shape or size. The balloon 402, once inflated, is pulled back toward the distal end of the catheter 401 until it engages the distal tip 220 of the infusion catheter at which point it is then aligned at the distal dialysis catheter 11 tip to ensure accurate placement. This process is shown in detail in FIG. 25, which illustrates stages A, B, C, and D of the devices at the distal tip. At stage A, a balloon tip is deflated and pushed past the distal end of the dialysis catheter. At stage B, the balloon tip is inflated as shown, and then at stage C, the infusion catheter 200 is retracted back into the dialysis catheter 11. Because the balloon 402 is inflated, it contacts the dialysis catheter 11 to establish proper placement prior to drug infusion. At stage D, the balloon tip 402 is deflated allowing drug infusion 50 to exit the catheter end 11. The drugs exit the infusion catheter 200 through the small openings or ports 279 which are, at stage D, inside the catheter end 11. The flow of the drug clears and dissolves the clot (not shown).

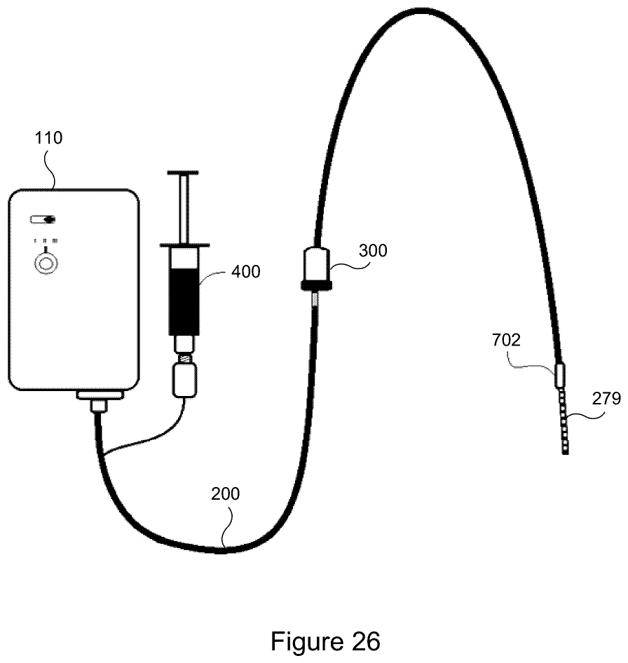

FIGS. 26, 27, and 28 discuss an alternative embodiment. FIG. 26 illustrates a distal balloon with distal infusion capability attached to a syringe. In this embodiment, the distal balloon 702 at the distal end of an infusion catheter 200 is inserted through a dialysis catheter (not shown). At the end of the infusion catheter 200 beyond the balloon 702 are numerous openings or ports through which a drug may be ejected or defused to the patient. An infusion system 110, as discussed above, contains the drug 50 for infusion to the patient. A syringe 400 includes a plunger that contains gas or liquid that is in fluid communication with the balloon 702.

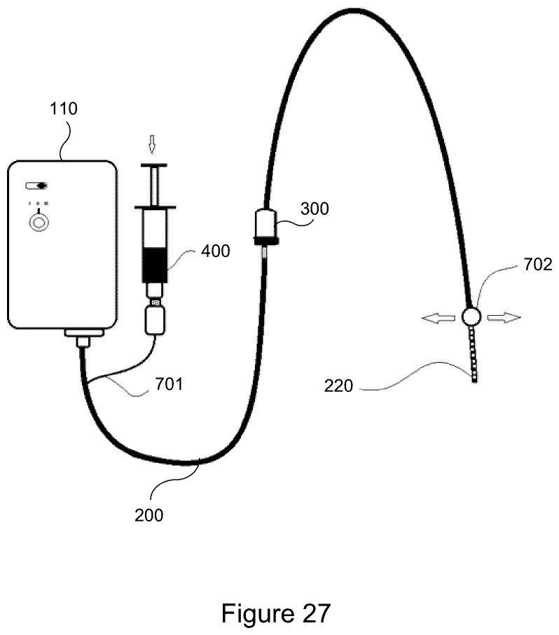

FIG. 27 illustrates an inflated distal balloon, inflated through use of a syringe, extending from a distal end of a catheter. During a subsequent stage, the plunger of the syringe 400 is depressed to inflate the balloon 702. In this embodiment and others, a separate air or fluid pathway may be provided for balloon inflation than is used for the infusion catheter's drug infusion. The balloon, after inflation, may then be pulled back to contact the end of the dialysis withdrawn to the end of the dialysis catheter as occurred in FIG. 25 to properly place the balloon at the end of the dialysis graft.

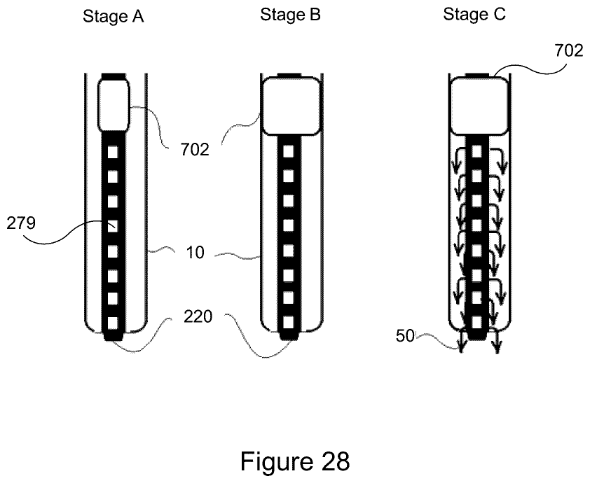

FIG. 28 illustrates an infusion catheter tip, at the distal end of a dialysis catheter, with the balloon deflated, prior to the balloon being drawn back into the dialysis catheter. In FIG. 28, at a stage A, after accurate placement as shown in FIG. 27, the balloon 702 is deflated and then drawn back into the dialysis catheter 11. The amount the balloon 702, and associated infusion catheter is drawn back into the dialysis catheter may vary subject to the intended function by the physician or nurse. In one embodiment, the balloon is withdrawing 2 centimeters into the distal end 220 of the dialysis catheter 11. In another embodiment, the balloon 702 may be withdrawing an amount equal to the infusion hole section of the infusion catheter. As a stage B, the balloon 702 is inflated to provide a full or partial seal (barrier) between the balloon and the wall of the dialysis catheter 11 to prevent the drug 50, when infused, from escaping back up into the dialysis graft. In this embodiment and others, the syringe 400 or any other device of mechanism may be used to inflate the balloon 702. Then, at stage C, the drug 50 is infused out of the openings 279, such as by depressing the plunger. Because of the inflated balloon 702 the drug 50 is forced out of the end of the dialysis catheter in a single direction outflow and cannot easily travel up into the dialysis catheter.

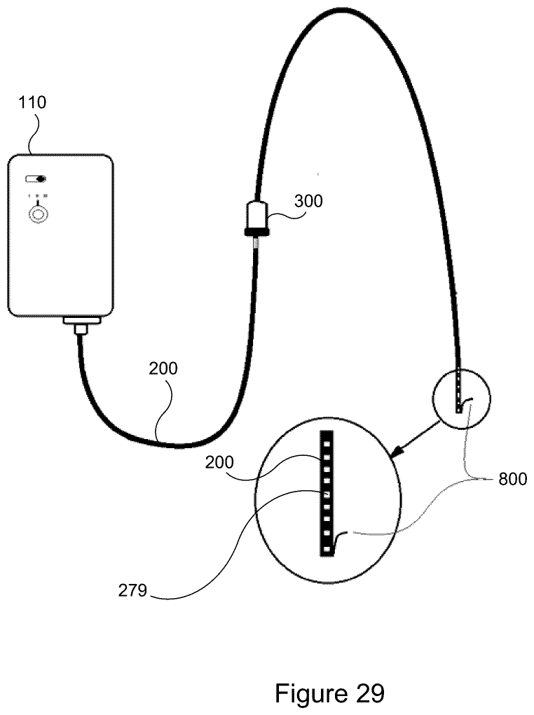

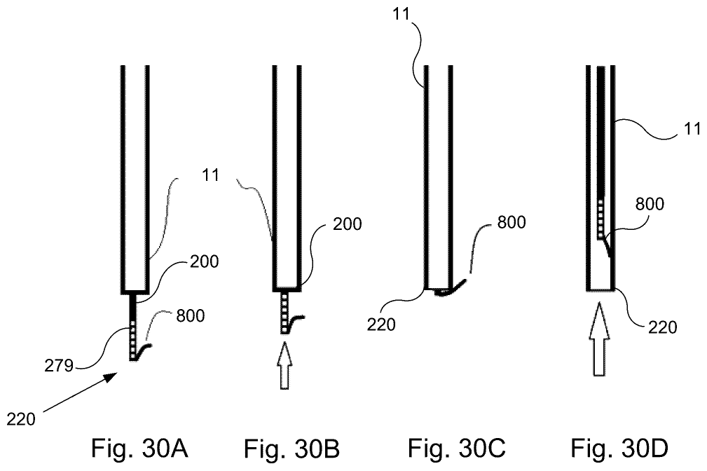

An additional embodiment is illustrated in FIGS. 29 and 30. FIG. 29 illustrates a barbed tip at the distal end of an infusion catheter which allows tactile resistance in catheter positioning. FIGS. 30A, 30B, 30C and 30D illustrates the barbed tip of the infusion catheter positioned at various locations distal to the dialysis catheter.

Drawing 30A shows the barbed tip 800 of the infusion catheter 200 positioned distal to the dialysis catheter 11. FIGS. 30B and 30C illustrates the infusion catheter 200 being pulled back with FIG. 30C showing the barb tip 800 engaging the distal end of the dialysis catheter 11 for correct positioning. FIG. 30D illustrates the retroflexed barb 800 position as the infusion catheter 200 is withdrawn.

In this embodiment, a barb 800 is on the distal tip of the infusion catheter 200. The barb 800 is flexible and flattens parallel to the wall of the distal catheter tip when advanced into the dialysis catheter 11 and then re-expands once it is passed beyond the distal tip as shown in FIG. 30A. The barb 800 may be made from any flexible material with memory, spring characteristics, or elastic properties to return to its expanded position when outside the dialysis catheter 11. One exemplary material is nitinol. Although referred to as a barb 800, the barb may be any type prong or extension from the infusion catheter 200 configured to or capable of functioning as a placement indicator for the infusion catheter.

As shown in FIG. 30B, the infusion catheter 200 is then pulled back from its proximal end until the barb 800 engages the distal end 220 dialysis catheter 11 as shown in FIG. 30C. The user (doctor or nurse) will feel this as increased tension on the infusion catheter 200 and this will then be the optimal position to begin the infusion with the side openings 279 at the tip of the dialysis catheter 11. Alternatively, from the known position of the infusion catheter 200 in relation to the dialysis catheter 11, the user can then move the infusion catheter in to or out of the dialysis catheter 11 of known amount to properly place the distal end of the infusion catheter for optimal drug delivery. Once the infusion is completed, as shown in FIG. 30D, the user pulls the infusion catheter 200 until the barb 800 retroflexes, allowing the infusion catheter to be removed. This embodiment allows the user to position the infusion catheter 200 correctly with the infusion side openings 279 (for drug infusion) at the desired medication deployment site by feeling resistance when pulling back and being able to detect the location of the infusion catheter in relation to the dialysis catheter 11. The infusion catheter is removed by overcoming additional resistance level.

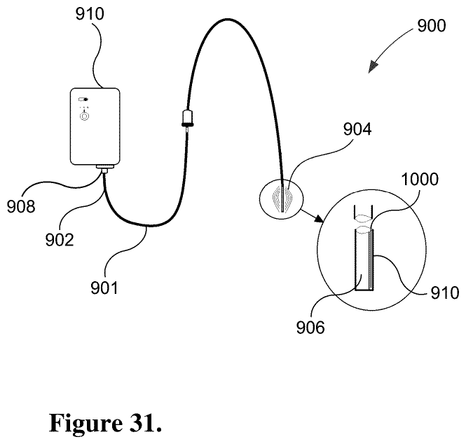

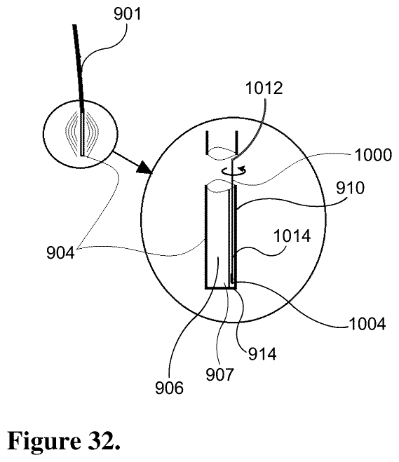

An additional embodiment is illustrated in FIG. 31, which shows a dialysis catheter clearance device 900, for use with a dialysis catheter. The device 900 includes an infusion catheter 901 having a first end 902, a second end 904, and a lumen 906 between the first end 902 and the second end 904. In one example, the infusion catheter 901 further includes a port 908 at the first end 902, and the port 908 is configured to connect to a medication delivery device 910 and accept clot dissolving medication that is provided to the port 908 from the medication delivery device 910. That is, the medication delivery device 910 may be configured to selectively output a clot dissolving medication. The catheter 901 may also include one or more medication outflow openings 907 (see FIG. 32) at the second end 904 of the infusion catheter 901. The outflow openings 907 have an aperture through which the clot dissolving medication exits the infusion catheter 901. The medication delivery device 910 may include any type of device or element that is capable of delivering medication into the infusion catheter. This includes, but is not limited to, a syringe, pump, and drip device.

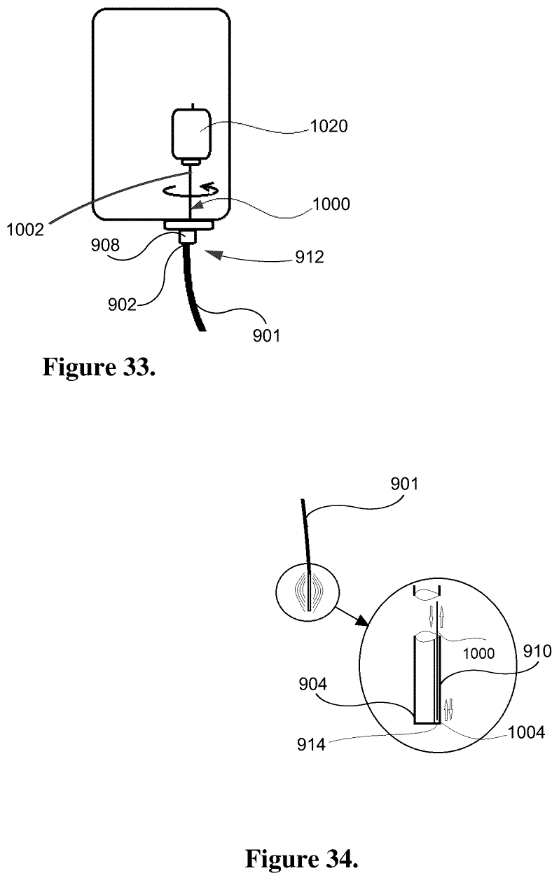

Additionally, as shown most clearly in FIGS. 32 and 33, the device 900 further includes a turbulence inducing element at the second end of the infusion catheter 901. Any type of turbulence inducing element may be utilized to impart motion, movement, or to excite the interaction between a clot and the medication used to dissolve the clot. In one embodiment, a flexible rod 1000 is provided as the turbulence inducing element, and has a first end 1002 and a second end 1004, with the first end 1002 being connected to a motion generating device (e.g., motor 1020, shown in simplified form in FIG. 33) of the device 900. While the device 900 is shown as employed with the motor 1020, other suitable alternative motion generating devices are contemplated, including piezoelectric type devices, vibrators, spinners, paddles/blades, or any other device.

In one example, the motor 1020 imparts movement to the rod 1000. The movement may be up and down in the direction of the catheter or rotational, or any other type motion. Furthermore, the second end 1004 of the rod 1000 is preferably located at or about the second end 904 of the infusion catheter 901. Moreover, in one example, actuation of the motor 1020 imparts movement to the second end 1004 of the rod 1000 in order to induce turbulence in the clot dissolving medication. As a result, the effectiveness of the clot dissolving medication is increased in dissolving a clot located at the second end 904 of the infusion catheter 901. More specifically, the vibration at the second end 904 of the infusion catheter 901 facilitates quicker clot lysis. As the medication is infused, the vibration at the second end 904 enhances the properties of the thrombolytic medication and shortens the lysis times.

In one particular example, the structure of the rod 1000 is advantageously configured to impart additional turbulence to the clot dissolving medication exiting the infusion catheter 901. Specifically, the rod 1000 preferably includes a wire portion 1012 extending from the first end 1002 of the rod 1000 to the second end 1004 of the rod 1000, and a weighted tip 1014 extending outwardly from the wire portion 1012 at the second end 1004 of the rod 1000. The weighted tip 1014 may be off-centered with respect to the wire portion 1012 (shown in FIG. 32), or may be centered with respect to a wire portion (not shown). By being off centered, additional turbulence may be imparted to the clot dissolving medication, such as if the rod is rotated and the off-center weighted tip induces vibration

Continuing to refer to FIG. 32, the infusion catheter 901 preferably has an internal sleeve 910 extending from at or about the first end 902 of the infusion catheter 901 to at or about the second end 904 of the infusion catheter 901. In one example the rod 1000 is located in the sleeve 910 from the first end 902 to the second end 904. However, it will be appreciated that in a suitable alternative arrangement of the disclosed concept, a flexible rod may be loosely located in a main central lumen of the infusion catheter 901 from the first end 902 to the second end 904.

Referring again to FIG. 33, actuation of the motor 1020 causes the rod 1000 to rotate in a manner wherein the second end 1004 (FIG. 32) of the rod 1000 remains located proximate the second end 904 of the infusion catheter while the motor 1020 is being actuated. Thus, when dialysis is being performed on a patient, and the infusion catheter 901 is inserted into a dialysis catheter, the motor 1020 causes the second 1004 to rotate within the second end 904, thereby causing the clot dissolving medication to more turbulently be delivered to a clot, and expedite lysis, etc.

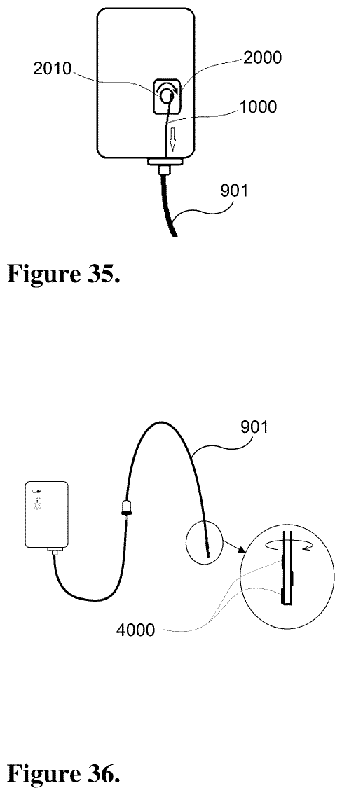

In another example, as shown in FIGS. 34 and 35, a motor 2000 (FIG. 35) optionally has a spool 2010 onto which the flexible rod 1000 is attached on an outer portion of the spool 2010, and actuation of the motor 2000 causes the rod 1000 to oscillate such that the second end 1004 of the rod 1000 moves toward and away from the second end 904 of the infusion catheter 901 while the motor 2000 moves the rod 1000. It will be appreciated that the sleeve 910 may have an open end (indicated generally with arrow 912 in FIG. 33) proximate the first end 902 of the catheter 901, and a closed end 914 at the second end 904 of the infusion catheter 901. Responsive to actuation of the motor 2000 (FIG. 35), the second end 1004 of the rod 1000 is configured to oscillate into and out of engagement with the closed end 914 of the sleeve 910. This oscillation and cyclical engagement between the rod 1000 and the closed end 914 is configured to impart turbulence to the clot dissolving medication as it exits the second end 904 of the infusion catheter. Stated differently, the repetitive release of energy by the second end 1004 causes the second end 904 (e.g., the tip) of the infusion catheter 901 to vibrate.

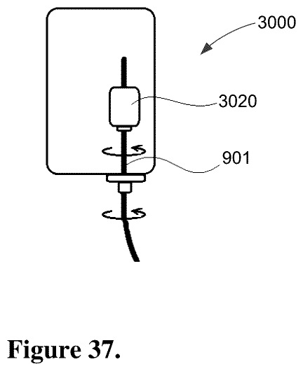

In one example, a motor is advantageously configured to cause the rod 1000 to vibrate, which may be a non-ultrasonic vibration level or ultrasonic vibration. In another example, as shown in FIG. 37, a dialysis catheter clearance device 3000 is configured such that responsive to actuation of a motion generating device, e.g., motor 3020, a second end of an infusion catheter 901 is configured to vibrate and/or turn the infusion catheter 901 within the dialysis catheter, thereby inducing turbulence to the clot dissolving medication at the second end of the infusion catheter 901. In such an example, the catheter 901 is directly coupled, e.g., either directly connected or via one or more intermediate components, to the motor 3020, such that the vibration, in this particular example, is imparted to the clot dissolving medication exiting the infusion catheter 901 without a rod or other turbulence generating device.

In another example, a dialysis catheter clearance device may include electrical wires connecting a vibrator or other device located at a second end of an infusion catheter, wherein such a device can be made to vibrate or otherwise impart motion using electricity as power.

In another example, as indicated in FIG. 36, coupled to an exterior of the infusion catheter 901 are a plurality of small paddles 4000. The small paddles 4000 will, responsive to rotation of the catheter 901, continuously mix the lytic medication enhancing its effectiveness. As shown in FIG. 37, the catheter 901 is connected to a rotational motor 3020, which turns the catheter 901 inside of the dialysis catheter. The small paddles 4000 could be molded into the catheter 901 or created by adhering small, raised material to the catheter 901.

It will also be appreciated that an example method of clearing a blood clot in a dialysis catheter includes a first step of providing a dialysis catheter clearance device 910, providing a turbulence inducing element 1000 (e.g., such as but not limited to a flexible rod) either together with the dialysis catheter clearance device 910 or independent thereof in a manner wherein the turbulence inducing element 1000 is inserted into a lumen 906 of the infusion catheter 901. This may include selectively connecting the turbulence inducing element 1000 to a motion generating device 1020 and 2000. The method may also include accessing an exposed opening of the dialysis catheter, inserting the second end 904 of the infusion catheter 901 into the exposed opening of the dialysis catheter, advancing the second end 904 of the infusion catheter 901 through the exposed opening of the dialysis catheter to place the second end 904 of the infusion catheter 901 at or near a clot that is blocking or inhibiting flow through the dialysis catheter. Additionally, the method may also include providing clot dissolving medication into the infusion catheter 901 to the second end 904 of the infusion catheter 901 or to a clot in the dialysis catheter, and activating the turbulence inducing element 1000 that is located at the second end 904 of the infusion catheter 901 with the motion generating device 1020 and 2000 in order to introduce turbulence in the medication, such that the medication exits the infusion catheter 901 in a turbulent manner which increases the rate at which the medication dissolves the clot.

In one example, activating the turbulence inducing element 1000 includes moving the turbulence inducing element 1000 independently with respect to the infusion catheter 901, and/or rotating the turbulence inducing element 1000 in a manner wherein the second end 1004 of the turbulence inducing element 1000 remains located proximate the second end 904 of the infusion catheter 901 while the turbulence inducing element 1000 is being activated. However, as indicated above with respect to FIGS. 34-35, activating the turbulence inducing element 1000 may include oscillating or rotating the turbulence inducing element 1000 with the motion generating device 1020 and 2000 such that the second end 1004 of the turbulence inducing element 1000 moves toward and away from the second end 904 of the infusion catheter 901 while the turbulence inducing element 1000 is being activated. Oscillating the turbulence inducing element 1000 may include moving the second end 1004 of the turbulence inducing element 1000 into and out of engagement with the closed end 914 of the internal sleeve 910. Finally, activating the turbulence inducing element 1000 may also include rotating or ultrasonically vibrating the turbulence inducing element 1000 with the motion generating device 1020 and 2000.

Other systems, methods, features, and advantages of the invention will be or will become apparent to one with skill in the art upon examination of the following figures and detailed description. It is intended that all such additional systems, methods, features, and advantages be included within this description, be within the scope of the invention, and be protected by the accompanying claims.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

What is claimed is:

1. A dialysis catheter clearance device, for use with a dialysis catheter, comprising:
an infusion catheter having a first end, a second end, and a lumen between the first end and the second end, the infusion catheter further comprising:
a port at the first end of the infusion catheter, the port configured to connect to a mediation deliver device and accept clot dissolving medication that is provided to the port from the medication delivery device;
a one or more medication outflow openings at the second end of the infusion catheter, the outflow openings having an aperture through which the clot dissolving medication exits the infusion catheter; and
a flexible rod, within the infusion catheter, having a first end and a second end, the first end of the rod connected to a motor, which imparts movement into the rod, and the second end of the rod located at or about the second end of the infusion catheter,
wherein actuation of the motor imparts movement to the second end of the rod to induce turbulence in the clot dissolving medication thereby increasing the effectiveness of the clot dissolving medication in dissolving a clot located at the second end of the infusion catheter.

2. The dialysis catheter clearance device of claim 1, wherein the infusion catheter has an internal sleeve extending from at or about the first end of the infusion catheter to at or about the second end of the infusion catheter, and wherein the rod is disposed in the sleeve from the first end to the second end.

3. The dialysis catheter clearance device of claim 2, wherein the actuation of the motor causes the rod to rotate in a manner wherein the second end of the rod remains disposed proximate the second end of the infusion catheter while motor is being actuated.

4. The dialysis catheter clearance device of claim 2, wherein the actuation of the motor causes the rod to oscillate such that the second end of the rod moves toward and away from the second end of the infusion catheter while the motor is being actuated.

5. The dialysis catheter clearance device of claim 4, wherein the sleeve has an open end proximate the first end of the infusion catheter, and a closed end at the second end of the infusion catheter, and wherein, responsive to actuation of the motor, the second end of the rod is configured to oscillate into and out of engagement with the closed end of the sleeve.

6. The dialysis catheter clearance device of claim 2, wherein the rod comprises a wire portion extending from the first end of the rod to the second end of the rod, and a weighted tip extending outwardly from the wire portion at the second end of the rod.

7. The dialysis catheter clearance device of claim 6, wherein the weighted tip is off-centered with respect to the wire portion.

8. The dialysis catheter clearance device of claim 1, wherein the rod is loosely disposed in the lumen of the infusion catheter from the first end of the infusion catheter to the second end of the infusion catheter.

9. The infusion catheter clearance device of claim 1, wherein, responsive to actuation of the motor, the rod is configured to ultrasonically vibrate.

10. A dialysis catheter clearance device, for use with a dialysis catheter, comprising:
a medication delivery device configured to selectively output a clot dissolving medication, the medication delivery device comprising a motion generating device;
an infusion catheter having a first end and a second end, the first end of the infusion catheter connected to the motion generating device, and the second end of the infusion catheter having an opening through which the clot dissolving medication exits the infusion catheter,
wherein, responsive to actuation of the motion generating device, the second end of the infusion catheter is configured to vibrate, thereby inducing turbulence to the clot dissolving medication at the second end of the infusion catheter.

11. A method of clearing a blood clot in a dialysis catheter comprising:
providing a dialysis catheter clearance device, said dialysis catheter clearance device comprising a medication delivery device and an infusion catheter, said medication delivery device configured to selectively output a medication, said medication delivery device comprising a motion generating device, said infusion catheter having a first end connected to said medication delivery device, and a second end;
providing a turbulence inducing element either together with said dialysis catheter clearance device or independent thereof in a manner wherein said turbulence inducing element is inserted into said infusion catheter, wherein providing said turbulence inducing element comprises selectively connecting said turbulence inducing element to said motion generating device;
accessing an exposed opening of the dialysis catheter;
inserting the second end of the infusion catheter into the exposed opening of the dialysis catheter;
advancing the second end of the infusion catheter through the exposed opening of the dialysis catheter to place the second end of the infusion catheter at or near a clot that is blocking or inhibiting flow through the dialysis catheter;
providing clot dissolving medication into the infusion catheter to the second end of the infusion catheter or to a clot in the dialysis catheter; and
activating the turbulence inducing element that is located at the second end of the infusion catheter with the motion generating device in order to introduce turbulence in the medication, such that the medication exits the infusion catheter in a turbulent manner which increases the rate at which the medication dissolves the clot.

12. The method of claim 11, wherein the turbulence inducing element is disposed in an internal sleeve of the infusion catheter from the first end of the infusion catheter to the second end thereof, and wherein activating the turbulence inducing element comprises moving the turbulence inducing element independently with respect to the infusion catheter.

13. The method of claim 12, wherein the turbulence inducing element has a first end and a second end, wherein the second end of the rod is disposed proximate the second end of the infusion catheter, and wherein activating the turbulence inducing element comprises rotating the turbulence inducing element in a manner wherein the second end of the turbulence inducing element remains disposed proximate the second end of the infusion catheter while said turbulence inducing element is being activated.

14. The method of claim 13, wherein the turbulence inducing element is a flexible rod.

15. The method of claim 12, wherein the turbulence inducing element has a first end and a second end, wherein the second end of the turbulence inducing element is disposed proximate the second end of the infusion catheter, and wherein activating the turbulence inducing element comprises oscillating the turbulence inducing element with the motion generating device such that the second end of the turbulence inducing element moves toward and away from the second end of the infusion catheter while the turbulence inducing element is being activated.

16. The method of claim 15, wherein the internal sleeve of the infusion catheter has an open end proximate the first end of the infusion catheter, and a closed end at the second end of the infusion catheter, and wherein oscillating the turbulence inducing element comprises moving the second end of the turbulence inducing element into and out of engagement with the closed end of the internal sleeve.

17. The method of claim 16, wherein the turbulence inducing element is a flexible rod.

18. The method of claim 11, wherein the turbulence inducing element is a flexible rod comprising a wire portion and a weighted tip extending outwardly from a distal end of the wire portion, and wherein the weighted tip is disposed at the second end of the infusion catheter.

19. The method of claim 18, wherein the weighted tip is off-centered with respect to the wire portion.

20. The method of claim 11, wherein activating the turbulence inducing element comprises ultrasonically vibrating the turbulence inducing element with the motion generating device.
